(19) United States
(12) Patent Application Publication
(10) Pub. No.: US 2025/0262431 A1
(43) Pub. Date: Aug. 21, 2025

(54) SYSTEMS AND METHODS FOR TRANSCLIVAL TUMOR-TREATING FIELDS

(71) Applicant: The Brigham and Women's Hospital, Inc., Boston, MA (US)

(72) Inventors: Walid Ibn Essayed, Boston, MA (US); Joshua Daniel Bernstock, Newton, MA (US)

(21) Appl. No.: 19/058,502

(22) Filed: Feb. 20, 2025

Related U.S. Application Data
(60) Provisional application No. 63/555,648, filed on Feb. 20, 2024.

(51) Int. Cl. A61N 1/36; A61N 1/05; A61N 1/08; A61N 1/378

(52) U.S. Cl. CPC A61N 1/36002; A61N 1/05; A61N 1/086; A61N 1/3787

(57) ABSTRACT

An implantable device comprises a first support plate; a plurality of electrodes disposed on an outer surface of the first support plate, and a second support plate configured to be removably attached to the first support plate, wherein the electrodes are configured to generate a tumor treating field within a body surface.

### CROSS-REFERENCE TO RELATED APPLICATIONS

This application claims priority to and the benefit of U.S. Provisional Patent Application No. 63/555,648, filed Feb. 20, 2024, the entire contents of which are herein incorporated by reference in their entirety.

### TECHNICAL FIELD

This disclosure relates to skull base implantations for the tumor fields generating electrodes, including the mechanism for deployment, fixation, and plug/unplugging.

### BACKGROUND

Diffuse intrinsic pontine glioma (DIPG), a type of diffuse midline glioma (DMG), is a highly fatal pediatric brainstem glioma without any current effective treatment option. DIPG carries an extremely poor prognosis, with 2-year survival rates of <10% despite the maximal radiation therapy. Comparative examples of tumor treating fields (TTF) have not been used to treat DIPG/DMG. Other comparative treatments were superficial and did not hit deeper regions of the brain such as the pons. Multiple chemotherapy and immunotherapy treatment modalities are currently on trial. The overall prognosis remains poor and further treatment options are needed.

### SUMMARY

These and other problems may be overcome by the systems and methods described herein.

Disclosed herein are systems and methods for the use of tumor fields for the treatment of DIPG using an implantable array in close proximity to the pons. Ceramic arrays allow placement through the nose, fixation to the skull base, and the possibility of plugging/unplugging to allow MRI compatibility.

According to one aspect of the present disclosure, an implantable device is provided. The implantable device comprises a first support plate; a plurality of electrodes disposed on an outer surface of the first support plate; and a second support plate configured to be removably attached to the first support plate, wherein the electrodes are configured to generate a tumor treating field within a body surface.

According to another aspect of the present disclosure, a method of implanting an implantable device is provided. The method of implanting an implantable device comprises creating an opening into the clivus of a patient; inserting the implantable device via the opening, wherein the implantable device includes a first support plate, a plurality of electrodes disposed on an outer surface of the first support plate, and a second support plate configured to be removably attached to the first support plate; and securing the implantable device to a skull base of the patient proximate the clivus.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an example in more detail than is necessary for a fundamental understanding of the teachings of the disclosures. In the drawings:

FIG. 1A shows an example illustration of a skull.

FIG. 1B shows an example illustration of a location of placement of the device and an example illustration of device placed.

FIG. 1C shows an example illustration of i) Axial CT brain scan demonstrating measurement of the interotid distance and ii) Sagittal CT head scan.

FIG. 2 is an exploded view of the implantable device.

FIG. 3A is an isometric view of the first support plate.

FIG. 3B is an isometric view of the second support plate.

FIG. 4 is a 3D rendering of a transnasal view of an example placement of a flexible array affixed to the clivus.

FIG. 5 is a 3D rendering of the transclival electrode implanted in the clivus through a caudal view and an endonasal view.

FIG. 6 is an illustration of an example insertion technique.

FIG. 7 is a (I) Proposed external array configuration demonstrating the clival array wiring exiting the nose in red; and (II) Sagittal view of the clival array placement with transnasal wiring.

FIG. 8 is an image of a device showing electrical leads.

FIG. 9 is a flowchart describing an example method of implantation of a device in accordance with various aspects of the present disclosure.

FIG. 10 is a flowchart describing an example method of operation of a device in accordance with various aspects of the present disclosure.

### DETAILED DESCRIPTION

In the following detailed description, reference is made to the accompanying drawings in which specific examples are shown by way of illustration. These examples are described in sufficient detail to enable those of ordinary skill in the art to practice the disclosure. It should be understood, however, that the detailed description and the specific examples, while indicating examples of embodiments of the disclosure, are given by way of illustration only and not by way of limitation. From this disclosure, various substitutions, modifications, additions rearrangements, or combinations thereof within the scope of the disclosure may be made and will become apparent to those of ordinary skill in the art.

Unless otherwise indicated, the various features illustrated in the drawings may not be drawn to scale. The illustrations presented herein are not necessarily intended to be actual views of any particular method, device, or system, but are merely idealized representations that are employed to describe various embodiments of the disclosure. Accordingly, the dimensions of the various features as illustrated may be arbitrarily expanded or reduced for clarity. In addition, some of the drawings may be simplified for clarity. Thus, the drawings may not depict all of the components of a given apparatus (e.g., device) or method. In addition, like reference numerals may be used to denote like features throughout the specification and figures.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

Unless otherwise specified or indicated by context, the terms “a,” “an,” and “the” mean “one or more.” As used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “only one of,” or “exactly one of.” For example, a list of “only one of A, B, or C” indicates options of: A, but not B and C; B, but not A and C; and C, but not A and B. In contrast, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more A, one or more B, and one or more C. Similarly, a list preceded by “a plurality of” (and variations thereon) and including “or” to separate listed elements indicates options of one or more of each of multiple of the listed elements. For example, the phrases “a plurality of A, B, or C” and “two or more of A, B, or C” indicate options of: one or more A and one or more B; one or more B and one or more C; one or more A and one or more C; and one or more A, one or more B, and one or more C.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, and unless otherwise specified, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “subject” may be used interchangeably with the term “patient” or “individual.” As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

Diffuse intrinsic pontine gliomas (DIPG), a subset of diffuse midline gliomas that are H3K27 altered, as classified by the fifth edition of the WHO's Classification of Tumors of the Central Nervous System (CNS), are highly fatal pediatric brainstem tumors for which there are no curative treatment options. As such, the prognosis remains dismal, with a 2-year survival rate of <10% despite maximal radiotherapy.

One treatment for high-grade gliomas is electrotherapy, a method in which tumor cells are exposed to low-intensity (1-3 V) intermediate frequency (100-500 kHz) electric fields capable of inhibiting tumor cell growth. There are multiple applications of intermediate frequency electrotherapy, including pulsed electric fields, which induce electroporation, and tumor-treating fields (TTF). TTF are alternating current electric fields which are created by externally fixed arrays on the scalp, the arrangement of which can be individualized. Without wishing to be bound to any one theory of action, it is believed that the disruption of mitosis in dividing malignant cells is the mechanism by which TTF affects the tumor. In one comparative example, intratumoral modulation therapy (IMT) is a derivative approach of TTF which involves the local delivery of low-intensity sinusoidal intermediate-frequency electric fields by means of implanting field-generating electrodes within targeted tissue.

However, the intra-axial implantation of electrodes (such as that performed in the comparative example) is associated with a number of non-negligible risks, including but not limited to an injury to surrounding eloquent structures, particularly in highly functional tissue such as the brainstem. In addition, permanently implanted intra-axial hardware poses its own associated limitations and risks, including infection, migration, and/or dysfunction, any of which may require revision surgeries and prolonged hospital stays. With available electrode technology, tissue coverage achieved by an IMT electrode is limited, which also would require the placement of multiple electrodes to cover larger lesions. Finally, another constraint of an internally implanted system is the question of magnetic resonance imaging (MRI) compatibility, as MRI is generally used in the management/monitoring of patients with brain tumors.

Due to such limitations, the clinical usage of tumor-treating fields (TTF) in CNS cancers has been confined to external systems in comparative examples, such as the transcranial scalp electrode system commercially available by Optune, in the management of glioblastoma (GBM). The results of TTF in GBM show a 2.7-month and 4.9-month increase in progression-free survival (PFS) and overall survival (OS) observed, respectively, when used in combination with current therapeutic standards. However, one of the inherent limitations affecting comparative examples in the setting of transcranial field generation is the depth of targeted tissue and the insulating effect of the skull. Clinical evidence has demonstrated that field intensity delivered to the site of the tumor is correlated with overall survival. Accordingly, other comparative examples of TTF utilize cranial remodeling interventions, in which the calvarial bone is either surgically thinned and/or burr holes or small craniectomies are strategically placed either during the initial tumor resection or re-resections with the aim of enhancing TTF penetrance and intensity.

There is little or no published data regarding the treatment of DIPG patients with TTF due in part to the various anatomical and engineering challenges defined above. Given in vitro data and the need for focused, non-toxic pediatric therapies, a deeper investigation of this modality is warranted. As such, the practicability of placing an implantable array in close proximity to the pons using a transnasal corridor was first clarified. This concept involves a hybrid approach taking advantage of the anatomical location of DIPG and is centered on the implantation of an intracranial extra-dural electrode embedded within the clivus, just ventral to the pons. To show this, anatomical feasibility of endoscopic endonasal placement of TTF electrodes in pediatric DIPG was evaluated. The feasibility of this approach represents an integral step in the process of developing a clival electrode form factor and investigating clival TTF as a treatment method for DIPG.

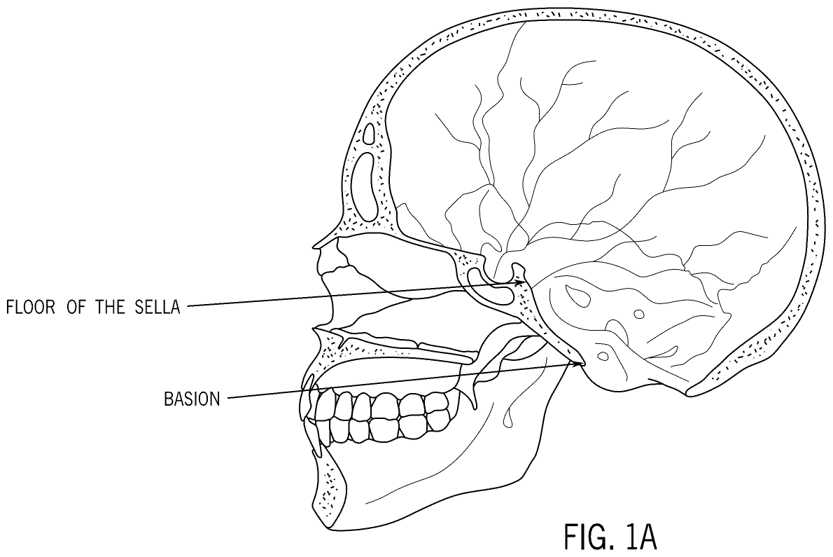

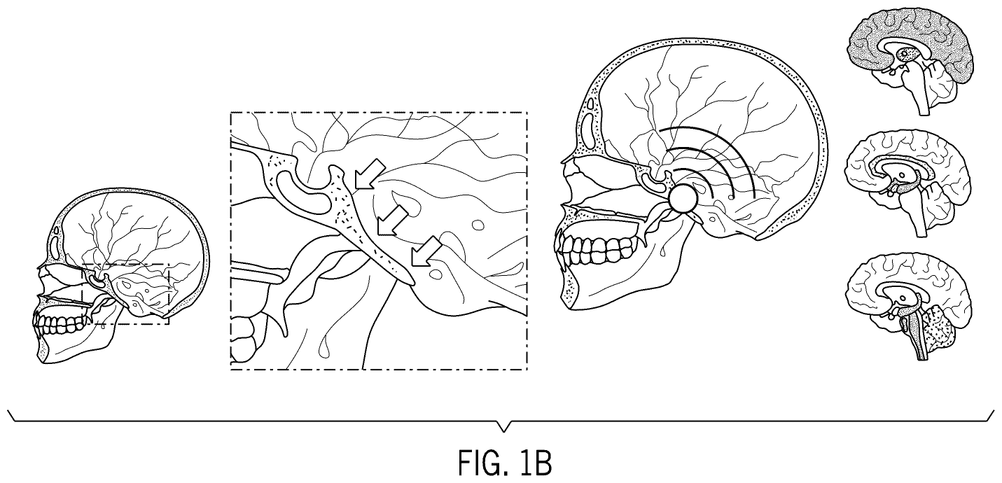

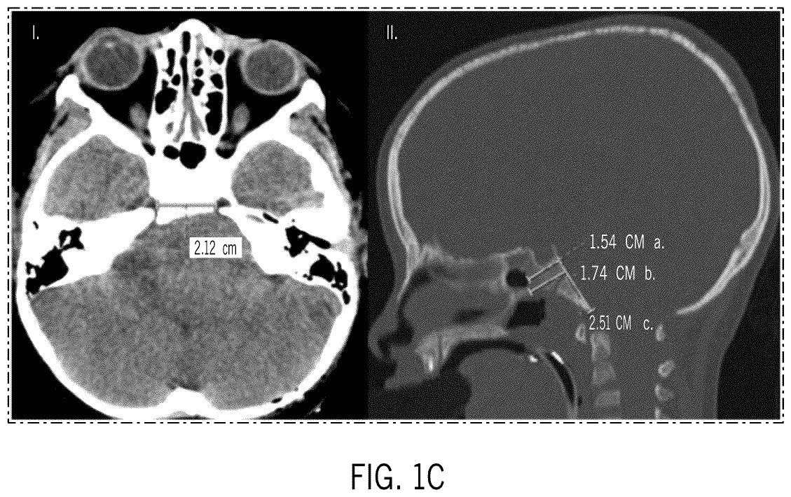

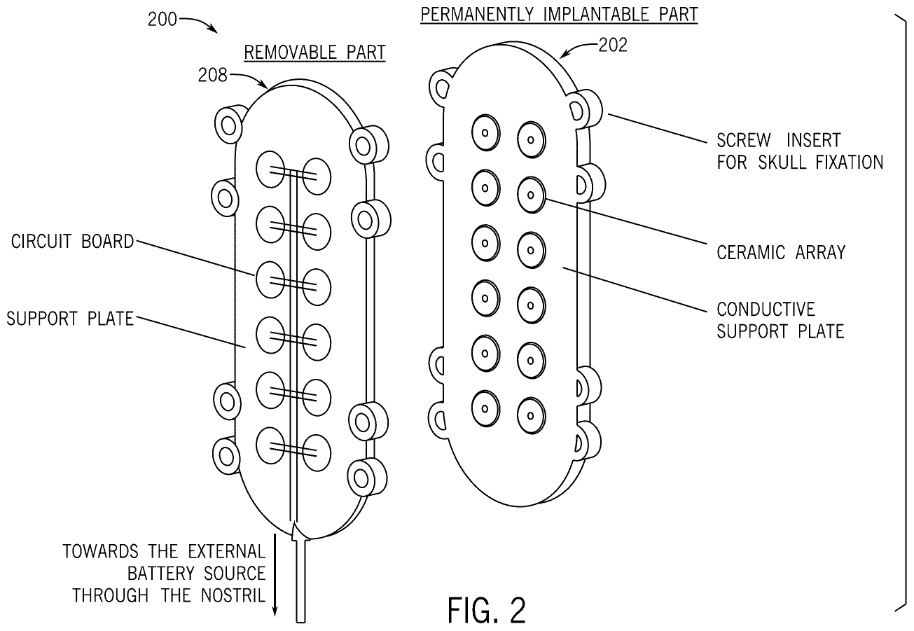

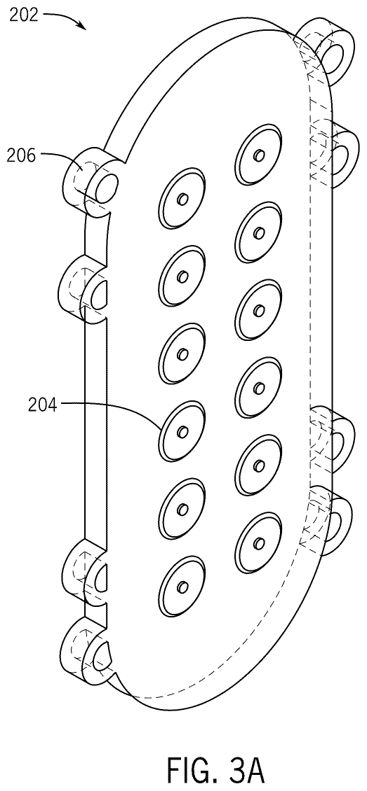

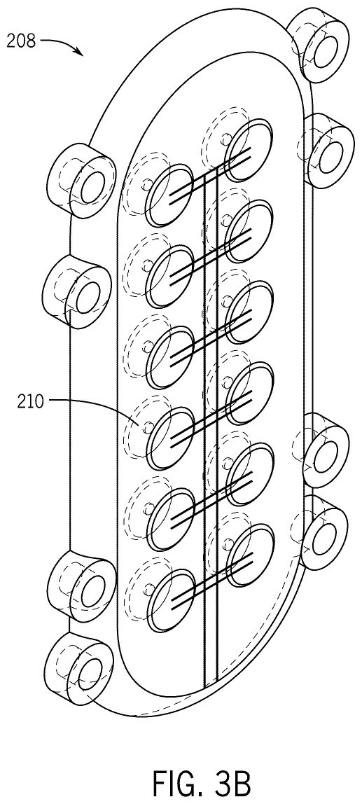

As can be seen in the illustration of FIG. 1A, the floor of the stella and basion are identified in the image of the skull. After clival partial or total drilling, the implantable device 200 (see FIG. 2) will be secured to the skull base, extending from the floor of the sella to the basion. The permanently implantable part of the implantable device 200 or first support plate 204 (as shown in FIGS. 2 and 3A) will be secured to the skull base using surgical titanium screws, allowing it to conform to the skull base anatomy extradurally, facing the pons.

Referring now to FIG. 1B, the location of implantation of the implanting device is illustrated by the arrows in the images on the left hand side. A graphical representation of the implantable device 200 in an implanted state is shown in the image on the right hand side.

Patients who presented with a clinical, radiographic, and histologically confirmed diagnosis of DIPG were retrospectively reviewed from 2010 to 2022. An algorithm was used to interrogate an institutional database using the keywords “diffuse intrinsic pontine glioma” or “DIPG” to identify patient charts. These charts were then manually reviewed to verify the diagnosis of DIPG and the presence of CT scans. Patients who did not have a CT scan were excluded. This protocol was reviewed by the local IRB under protocol number IRB-P00027869 and had an approved waiver of consent to conduct retrospective research.

The anatomical features that limit transclival approaches were characterized. The width of the approach was measured as the minimum lacerum inter-carotid distance. The degree of pneumatization of the sphenoidal sinus was divided into conchal, pre-sellar, sellar, and post-sellar. The height of the clivus was measured in the midsagittal plane extending from the base of the sella to the basion. The thickness of the clivus was measured at two levels: at the level of the sellar floor and at the thickest portion measurable on the midsagittal reconstruction (FIG. 1C).

Referring now to FIG. 1C, the scans illustrate: (I) an axial CT brain demonstrating measurement of the intercarotid distance, and (II) A sagittal CT head with bone window in the mid-sagittal plane demonstrating measurement of the thickness of the clivus (a) at the level of the sellar floor as well as (b) at the thickest portion measurable, and (c) the height of the clivus from sellar floor to basion.

Linear regression analyses were conducted to determine the relationships between age at the time of the CT and all anatomical measurements, including sphenoid pneumatization. The degree of pneumatization was then codified from 1-4 with 1 representing conchal, 2 pre-sellar, 3 sellar, and 4 post-sellar, for logistic regression analysis. All statistical analyses were conducted using StatPlus. A p-value of <0.05 was used to determine significance. The figures were created using BioRender.com or 3D slicer.

Forty-three unique patients were algorithmically identified as having a diagnosis of DIPG. Of these 43 patients, 17 patients had a verified tissue diagnosis of DIPG, and also had CT scans with sagittal/axial sequences of appropriate quality to complete the desired measurements. Of these patients, ten were male and seven were female. Ages ranged from 4-15 years; the median age was 6 years. Of sphenoid pneumatization categories, six patients fell within the conchal pneumatization type, five were pre-sellar, four were sellar, and one was a post-sellar type (Table 1). The average height of the clivus from the base of the sella to the basion was 3.34±0.39 cm. The thickness of the clivus from the base of the sella was on average 1.43±0.69 cm. The greatest thickness of the clivus was on average 1.62±0.19 cm. The average inter-carotid distance was 1.67±0.36 cm (Table 2). Age at the time of CT was found to be a statistically significant predictor of clival length (R²=0.568, p=0.0005), the width of clivus at the sellar floor (R²=0.372, p=0.009), and degree of sphenoid pneumatization (R²=0.605, p=0.0002). Age was not a significant predictor of the greatest width of clivus (R²=0.004, p=0.82), or inter-carotid distance (R²=0.006, p=0.76) (Table 3).

TABLE 1
Demographics

| Demographic | Number of Participants |
|---|---|
| Sex | |
| Male | 10 |
| Female | 7 |
| Age | |
| 4-7 | 10 |
| 8-11 | 5 |
| 12-15 | 2 |
| Sphenoid pneumatization | |
| Conchal | 6 |
| Presellar | 5 |
| Sellar | 4 |
| Postsellar | 1 |

TABLE 2
Anatomical Measurements

| | Average | Standard Deviation |
|---|---|---|
| Clival length | 3.34 | 0.39 |
| (floor of sella to basion) | | |
| Width of clivus at sellar floor | 1.43 | 0.69 |
| Greatest width of clivus | 1.62 | 0.19 |
| Intercarotid distance | 1.68 | 0.36 |

TABLE 3
Relationship between age and anatomical measurements

| | R² | p-Value |
|---|---|---|
| Clival length | 0.568 | 0.0005 * |
| (floor of sella to basion) | | |
| Width of clivus at sellar floor | 0.372 | 0.009 * |
| Greatest width of clivus | 0.004 | 0.82 |
| Intercarotid distance | 0.006 | 0.76 |
| Sphenoid pneumatization | 0.605 | 0.0002 * |

* indicates significance p ≤ 0.05.

Referring to FIG. 2, an exploded view of the implantable device 200 is shown. The implantable device 200 includes a permanently implantable part or first support plate 202 and a removable part or second support plate 208. The implantable device 200 can also include a power source. In some examples, the power source is an external battery connected to the second support plate 208 via a conduit (e.g., a transnasal conduit). In some examples, the power source is an implantable battery. In some examples, a combined thickness of the first support plate 202 and the second support plate 208 in a state of attachment to one another is between approximately 2 to 3 mm.

Referring to FIG. 3A, an isometric view of the first support plate 202 is shown. The first support plate 202 further includes a plurality of electrodes 204 disposed on an outer surface of the first support plate 202 and at least one attachment mechanism 206. The attachment mechanism 206 can be an insert for a fastener, such as a screw. The attachment mechanism 206 allows for fixation (e.g., permanent or semipermanent) to the skull of a patient. The plurality of electrodes 204 are configured to generate a tumor treating field within a body surface. In some examples, the plurality of electrodes 204 are arranged in an electrode array having a dimension of approximately 2.5 cm by 1 cm. In some examples, the plurality of electrodes 204 are evenly spaced (e.g., in a rectangular array). In some examples, the first support plate 202 is formed of a material that is compatible with magnetic resonance imaging. In some examples, the first support plate 202 or the plurality of electrodes 204 may include a ceramic material.

Referring to FIG. 3B, an isometric view of the second support plate 208 is shown. The second support plate 208 includes a locking mechanism (not shown) configured to removably attach to the first support plate 202. The exterior surface of the second support plate 208 further includes a circuit board 210. In one example, the locking mechanism may be mechanical.

TTFs represent a non-invasive biophysical approach to the management of different cancers, including pancreatic, ovarian, lung, and brain tumors, and may be used as a fourth treatment modality alongside surgery, chemotherapy, and radiation. TTF for GBM involves applying alternating electric fields at intermediate frequencies and may require that the therapy be performed for greater than 18 hours per day in order to confer clinical benefit. While not wishing to be bound by any particular theory of operation, it is believed that multiple biomolecular mechanisms are involved in the antineoplastic effects of TTF. The antimitotic effect is the most widely accepted mechanism, yet other means have also been hypothesized and tested, including DNA-damage response, suppression of cancer cell migration, autophagy, innate immunity, and immunogenic cell death.

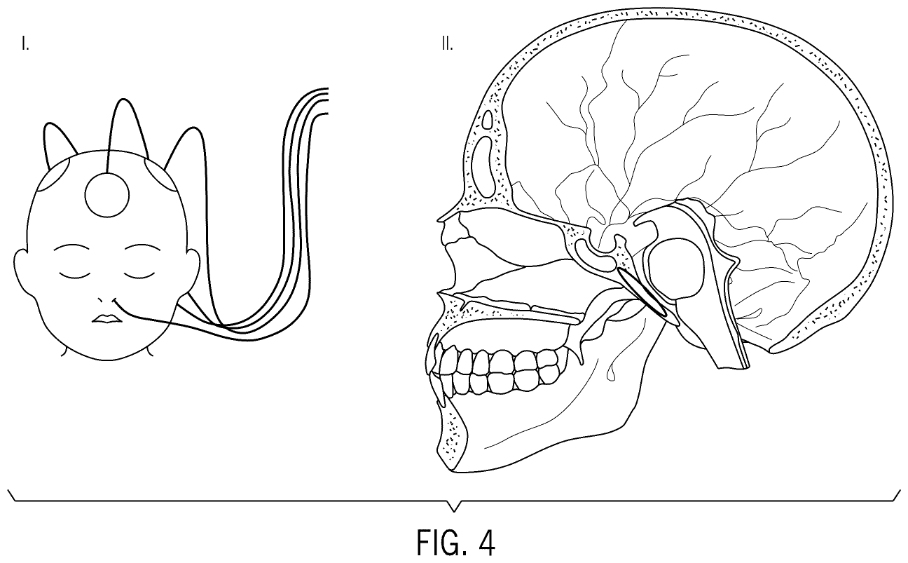

One limitation of comparative TTFs is the physical barrier of the surrounding soft tissue and skull which can lead to a significant dampening of field intensity, thereby decreasing treatment efficacy. Experimental modeling has shown the potential improvement of TTF efficacy by decreasing skull thickness through the strategic use of surgical thinning or bone-removal techniques. A phase I trial of Skull Remodeling Surgery (SR-Surgery) performed during the resection of the GBM demonstrated the safety of utilizing calvarial-thinning techniques along with multiple burr holes in and around the bone flap. The present disclosure employs these surgical remodeling techniques in the skull base and take advantage of the anatomical location of DIPG in the pons, just ventral to the clivus, via endoscopic implantation of an external TTF array within a surgically thinned clivus (FIG. 4).

Referring now to FIG. 4, (i) an example external array configuration demonstrating clival array wiring exiting the nose is illustrated in red and (ii) a sagittal view of the clival array placement with transnasal wiring are illustrated.

DIPG cells have been shown to be sensitive to low-frequency electrical stimulation in vitro as evidenced by reduced cell viability and increased apoptosis. When administered in combination with temozolomide (TMZ) and radiation therapy (RT), IMT combination therapy may be synergistic, achieving better results than monotherapy or dual TMZ-RT. Intratumoral or peritumoral electrode placement is limited due to the high degree of eloquence within the brainstem. Given the diffuse nature of pontine gliomas, it would be difficult to safely place enough electrodes in the parenchyma to provide sufficient coverage and produce a potential clinical benefit using comparative example systems and methods. TTF, which does not necessitate the placement in the parenchyma, permits treatment of eloquent brainstem lesions and has been demonstrated to have the depth of field penetration up to 30-40 mm, suitable for deeply located tumors.

In addition to the additive effects of TTF noted in combination with chemotherapy and radiation, TTF may also play an important role as an adjuvant treatment in immunotherapy trials by stabilizing tumor progression, thereby allowing patients to mount an immune response. This is particularly relevant given treatment modalities in the immunotherapy sphere for DIPG. TTF are capable of stimulating antitumor immunity properties, which may further produce a synergistic effect in combination with immunotherapies. As such, the combination of TTF with experimental treatment options may represent an exciting new avenue worthy of continued investigation.

The endoscopic endonasal implantation of a flexible TTF-generating electrode array in the clivus is presented herein as a treatment method for DIPG. Extended endoscopic endonasal approaches (EEA) have been extensively described in the literature. The anatomical limits of the transclival approaches have been rigorously studied in adults and are largely defined by the intercarotid distance at the level of the petrous apices as severe bleeding from a potential internal carotid artery injury represents one of the most serious risks. The sixth nerve, which runs along the clivus up towards the cavernous sinus from its exit point in the pons, can also be a limiting factor in extended EEA. When it comes to the pediatric population, EEAs are not as well understood due to the dynamic and variable anatomy within this cohort. Therefore, an assessment the size restrictions related to a planned clival implant in this specific age group of patients was performed. The patients in the study ranged from ages 4-15, with ˜60% falling between the ages of 4-7. Sphenoid pneumatization begins between the ages of 2-4 and is largely complete by age 12, although it is highly variable between individuals and can continue well into adulthood. The study confirmed that sphenoid pneumatization, clival length, and the width of the clivus at the sellar floor are significantly correlated with age. In our study population, the average maximum clival width was 1.62 cm. Given these findings, it is expected that younger patients may require more extensive drilling to place the implant at the optimal location on the face of the clivus with minimal intervening bone due to lower rates of pneumatization and thicker clival widths. Neuronavigation and Doppler ultrasound will, therefore, be tools to safely guide the procedure. The use of intraoperative CT may also be used to safely guide clival drilling. Augmented reality may be used as an adjunct technology that may also improve safe drilling practices, for example, by identifying and localizing the internal carotid arteries and overlaying the patient's anatomy in the endoscopic field of view. Of note, the intercarotid distance at the level of the clivus did not significantly correlate with age in the studied population. The study population had an average intercarotid distance of 1.68 cm, which would allow the safe implantation of an electrode array with a width of 1.5 cm in most patients.

Accessibility and sufficiency of the working corridor is another concern of pediatric endoscopic surgery, starting from the size of the nostril and including the size of the intranasal cavities. Finally, the vomer-clivus distance, unlike many of the other skull base dimensions, is not dependent on sphenoid pneumatization and does not change significantly during development, and therefore, should not pose an increased limitation in approach in children. Reconstruction is an additional consideration in pediatric EE surgery, as the nasoseptal flap, commonly used for reconstructions in this region, is insufficiently mature for transclival approaches. However, given that the procedure described herein is entirely extradural, reconstruction is generally not necessary. Preserving a thin layer of bone to protect the dura from inadvertent injury may represent a strategy to avoid accidental durotomies.

Overall, the results confirm that the implantation of a transducer array measuring 3×1.5 cm is feasible in 65% of patients due to limitations in either clival length or intercarotid distance, and that a 2.5×1 cm array would be implantable in 94% of patients studied. A consideration in the design of the transducer array is the ability to easily remove and replace the device, given that it is expected that this population of patients will require multiple MRIs over the course of their management. A detachable wiring system to allow the removal of the non-MRI compatible portion prior to scans is described herein. Such a procedure could be performed as an outpatient with minimal anesthesia, or under anesthesia concomitantly with a sedated MRI. In some examples, the array wiring can exit through the nasal cavity can be managed similarly to a nasogastric tube when the device is in use (FIG. 4).

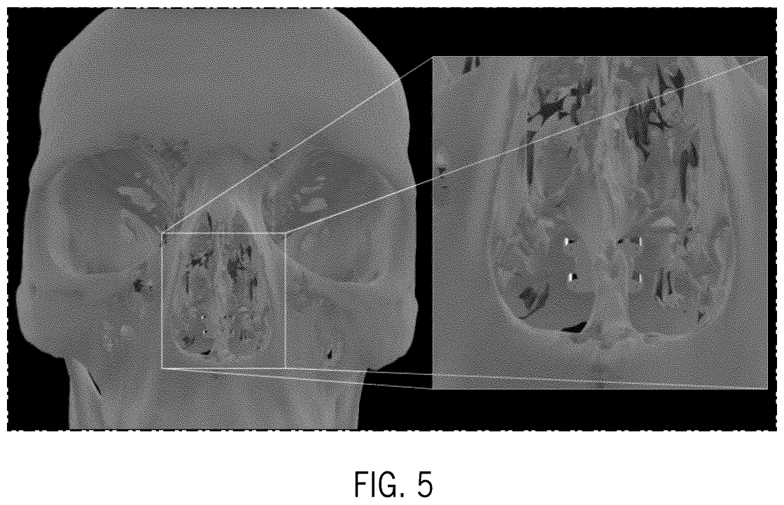

The anatomical feasibility of the transclival approach for TTF enhancement is confirmed. The next steps include the finite element modeling of the electrode array configuration for optimal pontine coverage. the potential efficacy of intracranial electrode arrays has been demonstrated. In doing so, configurations that universally required less current while still reaching significantly higher field strengths and therapeutic enhancement ratios (TER) in larger portions of the tumor bed as compared to transcranial controls have been defined. It has been posited that the desired transcranial array placement to achieve sufficient coverage in the brainstem would involve the placement of arrays on the vertex, bilateral posterolateral occiput, and superior-posterior neck. A transclival array may achieve adequate electric field distribution with minimal transcranial complementary arrays and less amperage, enhancing the practicality of the long-term use of the TTFs. Another consideration and active area of research is the material engineering of a flexible array that will permit insertion through the nose and fixation to the clival bone. This is illustrated in FIG. 5, in which a transnasal view of an example placement of a flexible array affixed to the clivus is shown.

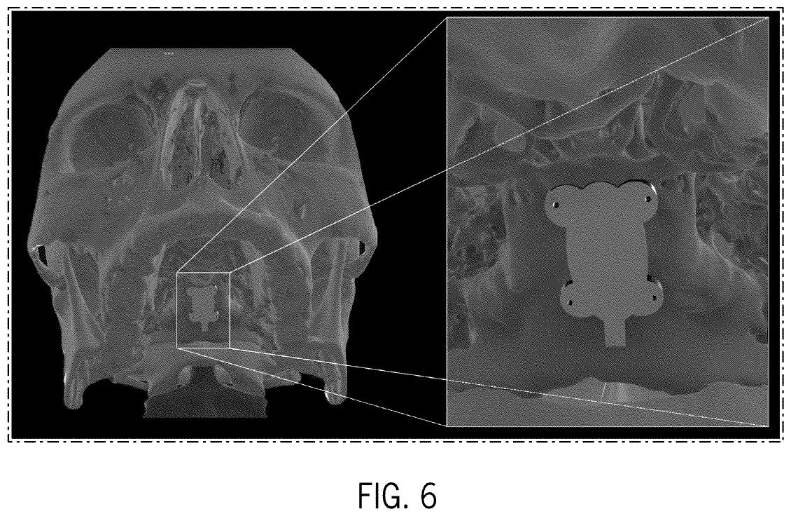

With reference to FIG. 6, the device may be an ovoid/rectangular shape with small lateral wings to facilitate anchoring the device to the clival bone (see, e.g., FIGS. 2-3B). The internal wiring of the array may be ceramic. In some embodiments, the internal wiring of the array may include an anti-microbial coating.

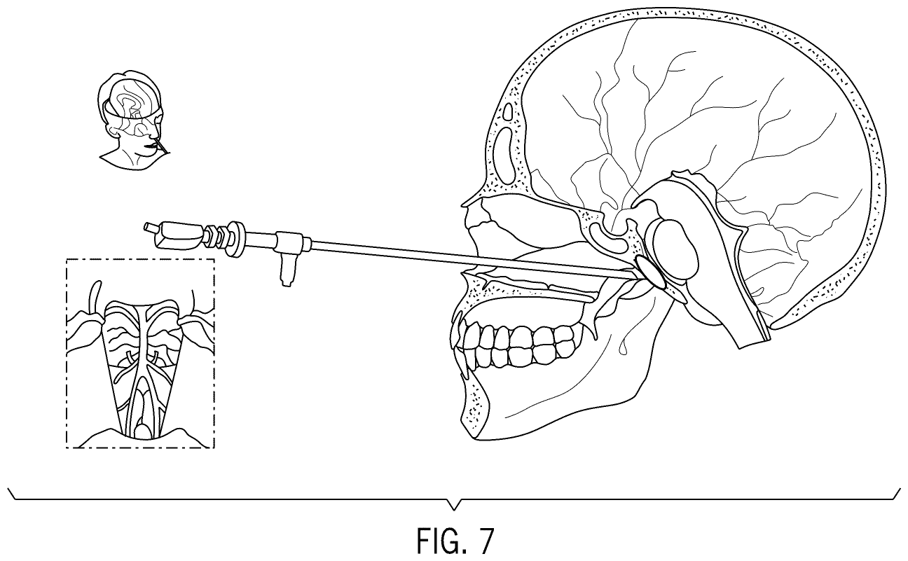

In the illustration of FIG. 7, an example insertion method is shown. In this insertion method, there is insertion through the nose. Insertion is followed by fixation to the clival bone. In some embodiments, there is a plug/unplug mechanism of the array wires to facilitate MRI scans. The insertion may be performed through the nose after drilling of parts of the skull base to accommodate the electrode array. This type of procedure is called craniopharyngiomas.

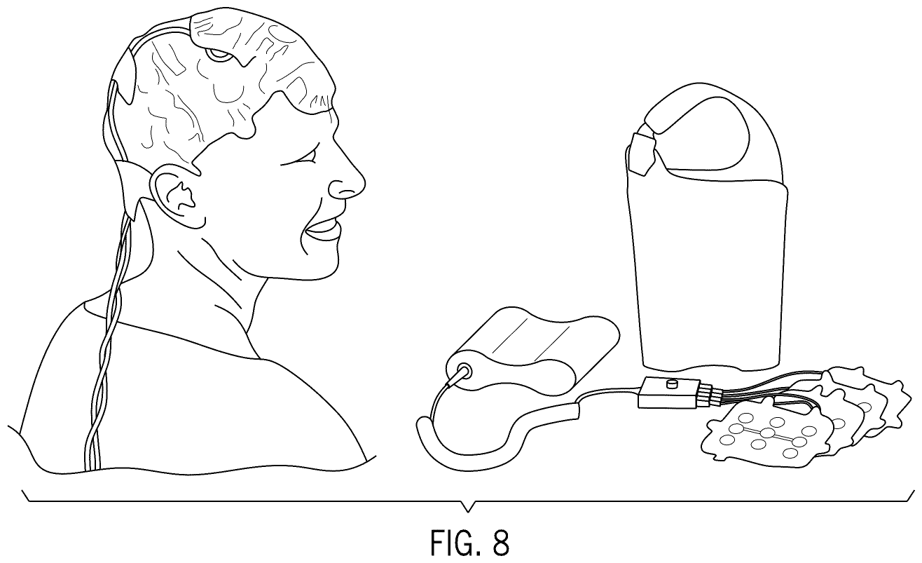

In the illustration of FIG. 8, example devices showing electrical leads and battery power are shown. The electrode array may be MRI compatible, as patients may require multiple MRIs as part of their oncological follow-up. The array itself can be MRI compatible, but in certain implementations the array may need to be unpluggable, which can be quickly done at the bedside by an otorhinolaryngologist prior to and after each MRI. The battery may be external, at the patient's waist, with the remaining of the array's battery. Externalization of the wiring can be done through the nose or the ear.

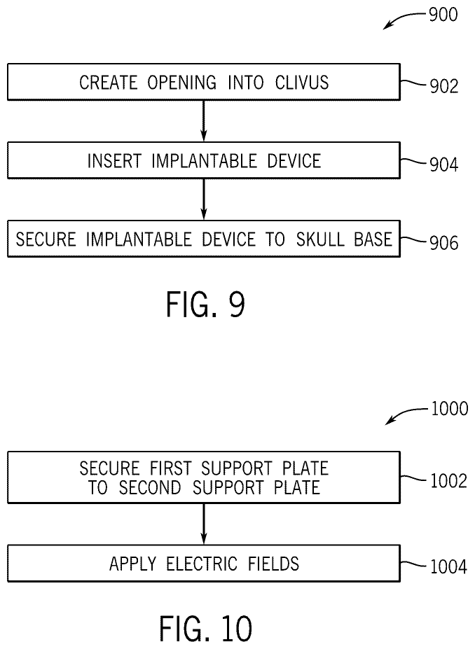

FIG. 9 illustrates an example method 900 of implantation of a device in accordance with the present disclosure. For purposes of explanation, the method 900 will be described as being performed using the implantable device 200 of FIGS. 2-3B; however, it should be understood that the method 900 may be performed using another implantable device in accordance with the present disclosure.

The method 900 begins with an operation 902 of creating an opening into a clivus of the patient. The opening may be created by, for example, drilling. Subsequently, at operation 904, the implantable device is inserted via the opening. As noted above, the implantable device may include a first support plate, a plurality of electrodes disposed on an outer surface (e.g., a brain-stem-facing surface) of the first support plate, and a second support plate configured to be removably attached to the first support plate. In some examples, operation 904 may include inserting the entirety of the implantable device through the opening; however, in other examples, operation 904 may include inserting only a portion of the implantable device (e.g., the first support plate with electrodes thereon) though the opening. Next, at operation 906, the implantable device is secured to the skull base of the patient at a location proximate the clivus. In implementations wherein operation 904 includes inserting only a portion of the implantable device, operation 906 may be followed by an operation of inserting the remaining portion of the implantable device (e.g., the second support plate) through the opening and attaching the remaining portion to the originally inserted portion.

FIG. 10 illustrates an example of a method 1000 of operating a device in accordance with the present disclosure. For purposes of explanation, the method 1000 will be described as being performed using the implantable device 200 of FIGS. 2-3B that has been implanted using the method 900 of FIG. 9. However, it should be understood that the method 1000 may be performed using an implantable device other than the implantable device 200 and/or using an implantable device that has been implanted using a method other than the method 900.

The method 1000 begins, in some implementations, with an operation 1002 of securing the first support plate to the second support plate. However, if the first support plate has already been secured to the second support plate, operation 1002 may be omitted from the method 1000. In either case, in a state of the first support plate and the second support plate being attached to one another (i.e., with the implantable device fully implanted and assembled), the method 1000 includes an operation 1004 of applying electric fields. Operation 1004 may include generating a TTF within a body surface of the patient via the plurality of electrodes. Operation 1004 may include providing power to the plurality of electrodes form a power source, which may be attached to the second support plate via a transnasal conduit and/or may be implanted in the patient.

Endoscopic endonasal transclival implantation of a TTF array is anatomically feasible in the vast majority of pediatric DIPG patients assessed, and as such, may represent an adjuvant treatment for this intractable disease.

Other examples and uses of the disclosed technology will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

What is claimed is:

1. An implantable device, comprising:
a first support plate;
a plurality of electrodes disposed on an outer surface of the first support plate; and
a second support plate configured to be removably attached to the first support plate,
wherein the electrodes are configured to generate a tumor treating field within a body surface.

2. The implantable device according to claim 1, wherein the second support plate has a locking mechanism configured to removably attach to the first support plate.

3. The implantable device according to claim 2, wherein the locking mechanism is mechanical.

4. The implantable device according to claim 1, wherein the first support plate and the second support plate further include at least one attachment mechanism.

5. The implantable device according to claim 4, wherein the at least one attachment mechanism is an insert for a fastener.

6. The implantable device according to claim 1, wherein the second support plate further comprises a circuit board.

7. The implantable device according to claim 1, further comprising a power source.

8. The implantable device according to claim 7, wherein the power source is an external battery connected to the second support plate via a conduit.

9. The implantable device according to claim 7, wherein the power source is an implantable battery.

10. The implantable device according to claim 1, wherein the plurality of electrodes are evenly spaced.

11. The implantable device according to claim 1, wherein plurality of electrodes are arranged in an electrode array having a dimension of approximately 2.5 cm by 1 cm.

12. The implantable device according to claim 1, wherein the first support plate is formed of a material that is compatible with magnetic resonance imaging.

13. The implantable device according to claim 12, wherein at least one of the first support plate or the plurality of electrodes includes a ceramic material.

14. The implantable device according to claim 1, wherein a combined thickness of the first support plate and the second support plate in a state of attachment to one another is between approximately 2 to 3 mm.

15. A method of implanting an implantable device, comprising:
creating an opening into a clivus of a subject;
inserting the implantable device via the opening, wherein the implantable device includes a first support plate, a plurality of electrodes disposed on an outer surface of the first support plate, and a second support plate configured to be removably attached to the first support plate; and
securing the implantable device to a skull base of the subject proximate the clivus.

16. The method of claim 15, wherein the first support plate is a permanently implantable part that is configured to attach to the skull base extending from a floor of a sella of the skull to a basion of the skull.

17. The method of claim 15, further comprising generating a tumor treating field within a body surface via a plurality of electrodes.

18. The method of claim 17, wherein generating the tumor treating field includes providing power to the plurality of electrodes from a power source.

19. The method of claim 18, wherein the power source is attached to the second support plate via a conduit extending through a nostril of the subject.

20. The method of claim 18, wherein the power source is implanted in the subject.
